(19) United States
(12) Patent Application Publication
(10) Pub. No.: US 2023/0348943 A1
(43) Pub. Date: Nov. 2, 2023

(54) BIOPLASTIC PRODUCTION METHOD

(71) Applicant: Algae Consultant LLC, Leander, TX (US)

(72) Inventor: Rebekah Powell, Leander, TX (US)

(21) Appl. No.: 17/661,169

(22) Filed: Apr. 28, 2022

(51) Int. Cl. C12P 7/64; C12N 1/20; C12N 1/12; C08G 63/16; C12N 1/06; C12M 1/00; C12M 1/12; C12M 1/107; B01D 15/36; C02F 1/42; B01D 53/84

(52) U.S. Cl. CPC C12P 7/64; C12N 1/20; C12N 1/12; C08G 63/16; C12N 1/06; C12M 21/02; C12M 23/06; C12M 23/22; C12M 23/36; C12M 47/10; C12M 31/10; B01D 15/363; C02F 1/42; B01D 53/84; C02F 2001/422

(57) ABSTRACT

The present disclosure describes a manufacturing method to use algae as a renewable green factory for producing biodegradable bioplastic. One or more embodiments include separation of a cultivated microalgae biomass from water before use in the wet or dried state. The lipids and proteins are extracted from the biomass which leaves starch and algae precursors in the remaining material from the microalgae cells. The starch includes amylose, amylopectin, monosaccharides kinases and cyclobutadiene and is hydrolyzed into a syrup containing oligosaccharides and polysaccharides. In some cases, the syrup is used as an ingredient in a medium containing nutrient for bacterial fermentation of plastics.

### BACKGROUND

The following relates generally to bioplastic manufacturing, and more specifically to algae-based bioplastic production.

Conventionally, plastic materials are derived from petroleum or natural gas through human industrial systems. Plastics are synthetic or semi-synthetic materials that are made of polymers. Plastics are light-weight, durable, less expensive, and can be molded or extruded to change shapes. As a result, plastics are used in multiple industries, for example, packaging, automobile, furniture, paints and coatings etc.

Biobased products refer to substances produced by a biological product or renewable agricultural or forestry materials. For example, biobased products may include paper and packaging materials, landscaping materials, etc. In some cases, biobased products may include bio-based plastics that are derived from renewable raw materials. Bio-based plastics may have multiple applications including packaging.

Conventional plastic materials have a slow degradation rate in a natural ecosystem resulting in environmental concerns. In some cases, plastic materials may not be recycled causing pollution or landfill in water bodies such as oceans. High quantities (e.g., few hundred billion pounds) of petroleum-based plastics are produced annually which use petroleum oil (e.g., few million barrels a day) and generate slow decomposing waste (e.g., billion tons of plastic waste). Furthermore, existing methods of bio-plastic production are costly and inefficient. Therefore, there is a need in the art for systems and methods to produce bio-plastics efficiently.

### SUMMARY

The present disclosure describes systems and methods for the production of bioplastics from bacterial fermentation obtained from algae biomass and algae produced precursors. Embodiments of the disclosure include methods, solvents, reagent-based solutions from algae biomass to form a feed bioplastic producing bacterial fermentation that produces bioplastics.

A method, apparatus, and system for an algae to bioplastic production method are described. One or more aspects of the method, apparatus, and system include cultivating algae to produce a bacterial growth medium; fermenting the bacterial growth medium to produce an organic precursor compound including monomers or joined-together monomers; and inducing a chemical reaction to convert the organic precursor compound into a bioplastic material.

An apparatus, system, and method for an algae to bioplastic production method are described. One or more aspects of the apparatus, system, and method include an algae growth environment configured to produce a bacterial growth medium, and algae plastic precursor by cultivating algae; a fermentation component configured to produce an organic precursor compound including monomers or joined-together monomers by adding heat and bacteria to the bacterial growth medium; and an extraction component configured to convert the organic precursor compound into a bioplastic material.

### BRIEF DESCRIPTION OF THE DRAWINGS

FIG. 1 shows an example of a bioplastic production method according to aspects of the present disclosure.

FIG. 2 shows an example of a bioplastic production method (swim) according to aspects of the present disclosure.

FIG. 3 shows an example of a bioplastic production apparatus according to aspects of the present disclosure.

FIG. 4 shows an example of algal biomass pre-processing flowchart according to aspects of the present disclosure.

FIG. 5 shows an example of a method for an algae to bioplastic production method according to aspects of the present disclosure.

FIG. 6 shows an example of individual tube according to aspects of the present disclosure.

FIG. 7 shows an example of water flow diagram according to aspects of the present disclosure.

FIG. 8 shows an example of algae condensing diagram according to aspects of the present disclosure.

FIG. 9 shows an example of liquid/liquid separation tank according to aspects of the present disclosure.

FIG. 10 shows an example of an algae cultivation unit according to aspects of the present disclosure.

FIG. 11 shows an example of a light blocking barrier according to aspects of the present disclosure.

FIG. 12 shows an example of aa deionization apparatus according to aspects of the present disclosure.

### DETAILED DESCRIPTION

The present disclosure describes systems and methods for production of bioplastics. Embodiments of the present disclosure include an apparatus that uses algae as a renewable green feedstock for producing bioplastic material. The apparatus for bioplastic production extracts lipids and proteins from algal biomass and hydrolyzes the starch material remaining in the algae into a syrup comprising saccharide polymers (e.g., oligosaccharides, polysaccharides). The syrup may be used as an ingredient for bacterial fermentation of bioplastic products. The bioplastic production may be facilitated using algae and bacterial precursors in the formulation of bioplastic material.

Conventional techniques for bacterial fermentation for generating bioplastic materials use a source of nutrients for the bacteria to grow. In some cases, waste sugar sources are used as hydrolyzed starch to reduce the cost of bioplastic production. For example, waste sugar sources may include molasses, rice starch, sugar beets, corn, etc. However, bioplastics produced using food based biological starch have low production volume due to the low availability of sugar sources. In some cases, crops that provide waste sugar are not able to compare to the production volume of petroleum oil and thus, consumer demand for plastics.

As such, some manufacturing biodegradable bioplastics use a source of nutrients (e.g., hydrolyzed starch) for the bacteria to grow. In some cases, renewable waste sugar sources (e.g., molasses, rice starch, sugar beets, corn, etc.) are used for bioplastic production. However, use of food based biological starch to produce plastic results in production of only a small fraction of the total amount of plastic produced. The renewable bioplastics produced have small production volume due to the low availability of food-based starch or sugar sources. As a result, bioplastics do not replace consumer demand for single use petroleum plastic products which are produced on a large scale. Some aspects of the present disclosure provide for plastic to be produced at a scale competitive to petroleum plastic products. Therefore, embodiments of the present disclosure provide for a transformation of the plastic industry to become biodegradable at a large commercial scale.

By applying the technique for production of bioplastic materials, embodiments of the present disclosure provide an alternative to conventional plastic materials that are environmentally safe. The techniques for bioplastic production include large-scale (i.e., commercial level) apparatus for algae cultivation and condensation. One or more embodiments of the disclosure include a system of open tubular photobioreactor for algae cultivation that is energy efficient and not impacted by external environmental factors. In some cases, the photobioreactor system is monitored by real-time sensors. Additionally, robots and collaborative robots (i.e., cobots) are used for processing data generated by the sensors.

One or more embodiments of the present disclosure include intracellular complex sugar formation in an algae cell. The reaction is catalyzed by a glycosyltransferase. The sugar added is in the form of an activated sugar nucleotide. In some cases, the reaction may proceed with retention or inversion of configuration at the glycosidic carbon atom which results in formation of a new bond.

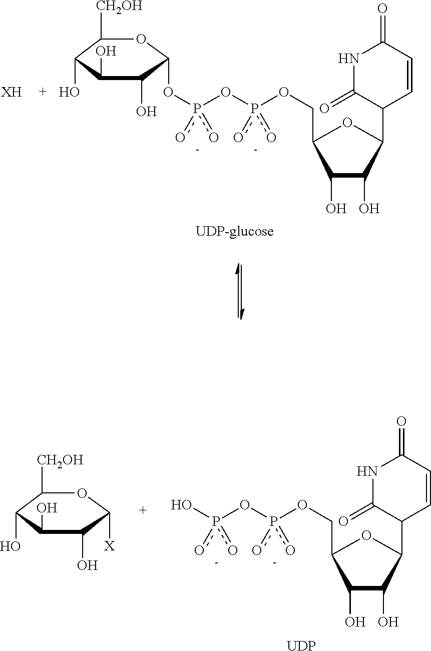

An embodiment of the present disclosure includes a wide array and variety of monosaccharide linkages which provide for multiple isomeric forms in complex carbohydrates. For example, two D-glucose residues are joined by a glycosidic linkage between the α-anomeric form of C-1 on one sugar and the hydroxyl oxygen atom on C-4 of the adjacent sugar. The glycosidic linkage is called an α-1,4-glycosidic bond. In some cases, monosaccharides have multiple hydroxyl groups which results in multiple glycosidic linkages. The array of the linkages with monosaccharides and the multiple isomeric forms of monosaccharides make complex carbohydrates.

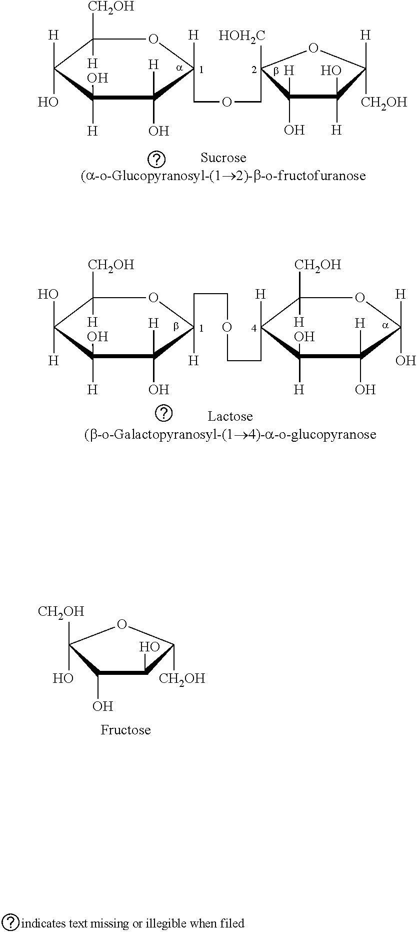

Hydrolysis of complex carbohydrates, or breaking the glycosidic linkages between glucose and fructose, to form simple sugar monomers may be performed with acid and/or enzymatic hydrolysis. Glucose may be the basis of the sugar bacterial syrup fermentation, and the fructose is purified and used in the medium to form a sugar source for the algae to grow. The hydrolysis is the basis for growing the bacteria and the algae. Fructose is not the only sugar used in fermentation. Other sugar monomers or polymers can be used to replace fructose. As long as the bacteria or algae can assimilate the monomer or polymer sugar source as energy to make the algae or bacterial precursors.

### Acid Hydrolysis of Sucrose into Glucose and Fructose

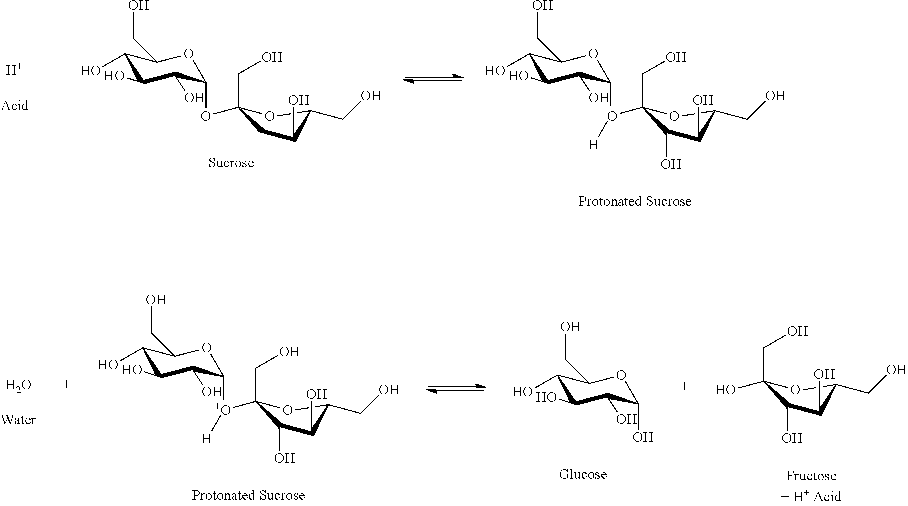

### Bioplastic Production Method

A method, apparatus, and system for one or more algae to bioplastic production methods are described. One or more aspects of the method, apparatus, and system include cultivating algae to produce a bacterial growth medium; fermenting the bacterial growth medium to produce an organic precursor compound including monomers or joined-together monomers; and inducing a chemical reaction to convert the organic precursor compound into a bioplastic material.

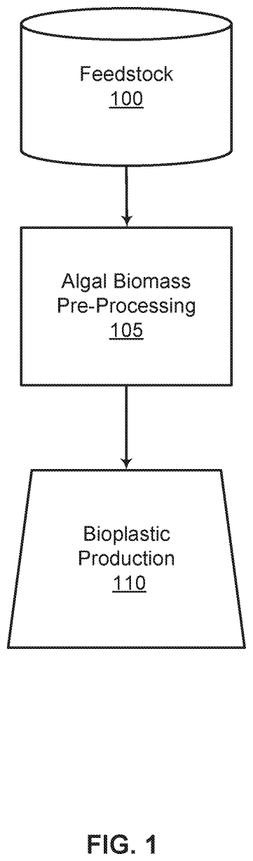

FIG. 1 shows an example of a bioplastic production method according to aspects of the present disclosure. The example shown includes multichambered feedstock 100 and algal biomass pre-processing 105, and bioplastic production 110. Feedstock 100 may then be used for algal biomass pre-processing 105, and the processed biomass may be used in bioplastic production 110.

Non-toxic algae may be used as feedstock 100. For example, algae that produce high concentrations of polysaccharides may be used. In some cases, algae produced in wastewater may be used as feedstock 100. Starch is a natural polymer available from multiple sources, for example, potato, corn, rice, tapioca, algae, etc. The intracellular concentration of the starch (i.e., 30%-50% wt.) includes amylose and amylopectin. The concentration of lactic acid is high in some living bacterial biomass species with a high rate of fermentation from the algae syrup and added nitrogen nutrients. Algae harvested directly from nature may include toxic species of algae. Therefore, algae sourced for bioplastic use are cultivated in a controlled growing environment and tested for the presence of neurotoxins. If algae are sourced from natural or artificial water sources a method has been developed to remove the toxic algae and verified to show that the toxins are no longer present.

One or more embodiments of the present disclosure modify starch into a syrup for fermentation. The algal biomass pre-production processing may use a bacterial nutrient syrup including the starch. The starch is modified using acid hydrolysis and/or enzymatic hydrolysis. Hydrolysis may facilitate the splitting of chemical bonds in starch leading to the formation of less complex sugars for bacterial digestion (e.g., oligosaccharides, polysaccharides). The process of hydrolysis is known to those skilled in the art.

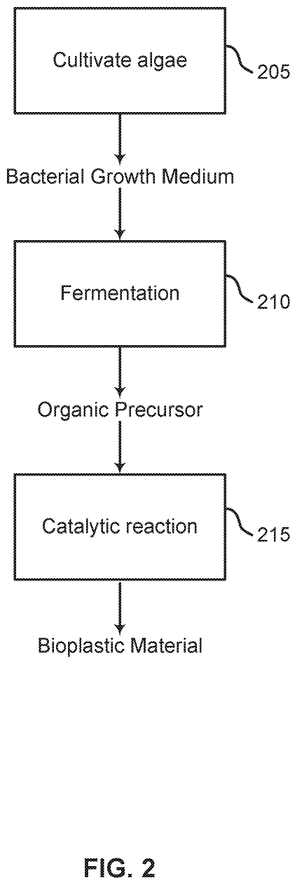

FIG. 2 shows an example of a method 200 of bioplastic production method according to aspects of the present disclosure. In some examples, these operations are performed by a system including a processor executing a set of codes to control functional elements of an apparatus. Additionally or alternatively, certain processes are performed using special-purpose hardware. Generally, these operations are performed according to the methods and processes described in accordance with aspects of the present disclosure. In some cases, the operations described herein are composed of various substeps, or are performed in conjunction with other operations.

Algae is passed through a redox reaction that separates the water from the algae. Redox reaction occurs when the algae interacts with the anion exchange causing a chain reaction that moves the algae away to the top of the water. In some embodiments, the ionized algae can be harvested or deionized to continue growth.

The catalytic reaction is of the form of enzymatic hydrolysis to break the bonds of the starch into simple sugars for the purpose of modifying the starch into a syrup for bacteria and other organisms for fermentation and cultivation.

At operation 205, the system cultivates algae. One or more embodiments of the present disclosure use algae that produce starch material to form a syrup solution and meet state and local standards for scientific and commercial cultivation. In short, the present disclosure modifies starch into a syrup for fermentation and cultivation. Algae are cultivated or skimmed from nature in higher concentrations to obtain a high wet or dry weight, slurry, or paste of algae for processing.

In some cases, commercial algae cultivation is performed using open cultivation and closed cultivation modes. However, conventional photobioreactors are shaped as racetrack cultivation pool or circular cultivation pool. Conventional photobioreactors include low efficiency of light energy utilization, high impact by external environmental elements, easy contamination, and large moisture evaporation.

At operation 210, the system performs fermentation. Fermentation is a sterile process optimized based on the output or concentration of lactic acid. Single or multiple strains of bacteria combined with the algae starch syrup are tested to optimize the production of lactic acid isomers. The parameters tested are temperature, sterility level, change of nutrient formulation with product output, and scalability. Lactic acid in bacteria is obtained by lysing or rupturing bacterial cell walls by heating the fermentation medium and or adding a base to the liquid in the fermenter.

At operation 215, the system modifies a polylactic acid plastic molecular configuration. The catalytic reaction happens when the biphytoplankton extract is added to the lactic acid produced by the bacteria fermentation process (operation 210). The biphytoplankton extract is produced from operation 205. Where the biomass is lysed and the extraction of cyclobutadine and a form of kinase acts as the catalyst.

The catalytic reaction includes a biplankton extraction from the algae. The algae have enzymes that catalyze a chain reaction to form a molecular tubular shape. This shape is very strong plastic material.

### Bioplastic Production Apparatus

A method, apparatus, and system for an algae to bioplastic production method are described. One or more aspects of the method, apparatus, and system include cultivating algae to produce a bacterial growth medium; fermenting the bacterial growth medium and introducing algae enzymes and algae precursors to produce an organic precursor compound including monomers or joined-together monomers; and inducing a chemical reaction to convert the organic precursor compound into a bioplastic material.

The present disclosure describes production of renewable bioplastics algae and lactic acid. One or more embodiments of the present disclosure use starch from algae to produce a mixture of oligosaccharide and polysaccharide syrup for bacterial fermentation. Algae is used as a source of starch (i.e., amylose) and organic precursors used in bioplastic production.

One or more embodiments of the disclosure include an apparatus for extracting lactic acid from the bacteria which results in the formation of polylactic acid (PLA) bioplastic. In some cases, biodegradable bioplastics are individual or blends of polylactic acid (PLA), polyhydroxybutyalkanoates (PHA), polyhydroxybutyrates (PHB). Most bioplastics are a single or combination of the above plastics listed. The plastic described here is a combination of algae and bacterial precursors to form a stronger PLA.

Embodiments of the present disclosure include an open photobioreactor for algae cultivation, algae condensation, and a direct air capture of carbon dioxide (CO2) to sequester the CO2 in the air for large-scale algae production.

Embodiments of the present disclosure describe methods and materials for synthesis of bioplastic materials that are an alternative to petroleum-based plastic materials. One or more embodiments use edible algae to produce a food grade mixture that includes an oligosaccharide and polysaccharide syrup for bacterial fermentation. In some cases, the algae used as a source of starch provides the same starch found in conventional food-based sources of starch, for example, corn, rice, beets, etc. An embodiment of the present disclosure includes an apparatus for large-scale extraction of lactic acid from bacteria which results in formation of bioplastic (e.g., poly-lactic acid).

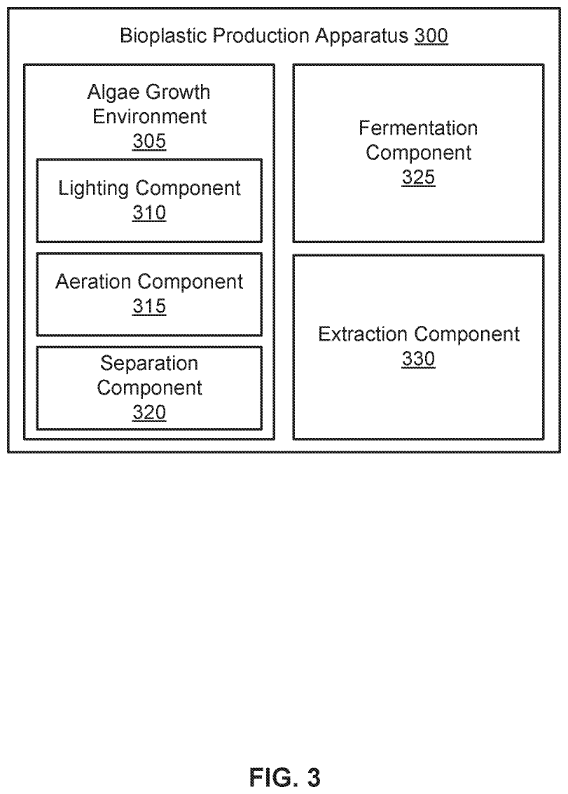

FIG. 3 shows an example of a bioplastic production apparatus 300 according to aspects of the present disclosure. In one aspect, bioplastic production apparatus 300 includes algae growth environment 305, fermentation component 325, and extraction component 330.

According to some aspects, algae growth environment 305 cultivates algae to produce a bacterial growth medium. In some examples, algae growth environment 305 produces the algae in an algae growth environment 305. In some examples, algae growth environment 305 hydrolyzes the algae to produce the bacterial growth medium. Additionally, or alternatively, the algae growth environment 305 is configured to produce a bacterial growth medium by cultivating algae. In some aspects, the algae growth environment 305 includes a set of transparent tubes. Algae is a unique organism that produces many types of products.

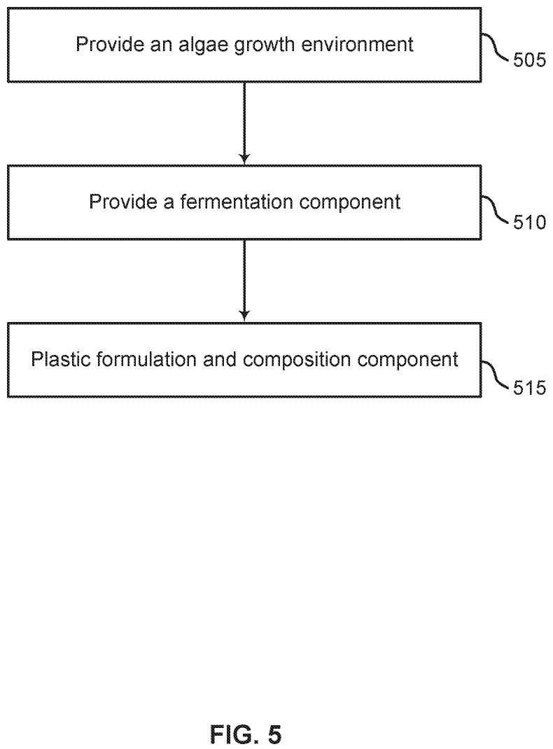

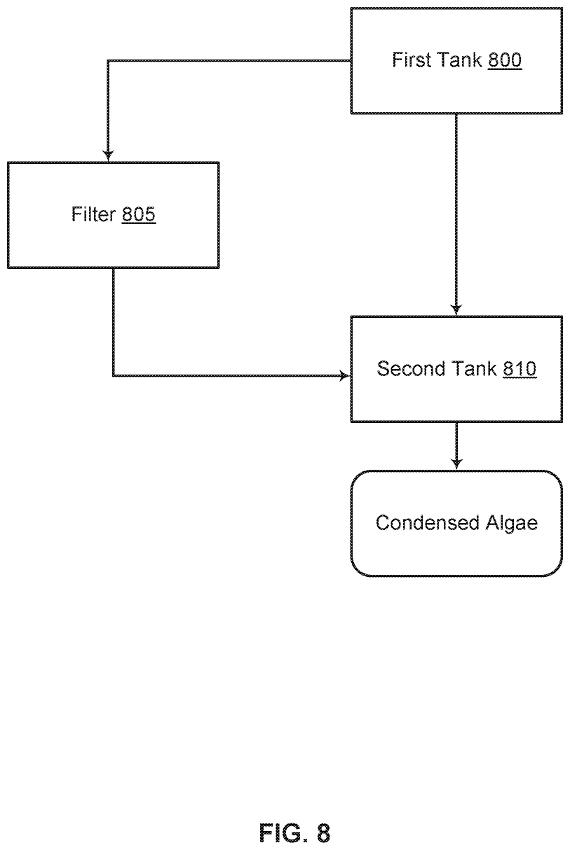

Algae growth environment 305 is an example of, or includes aspects of, the corresponding element described with reference to FIGS. 5 and 8. In one aspect, algae growth environment 305 includes lighting component 310, aeration component 315, and separation component 320.

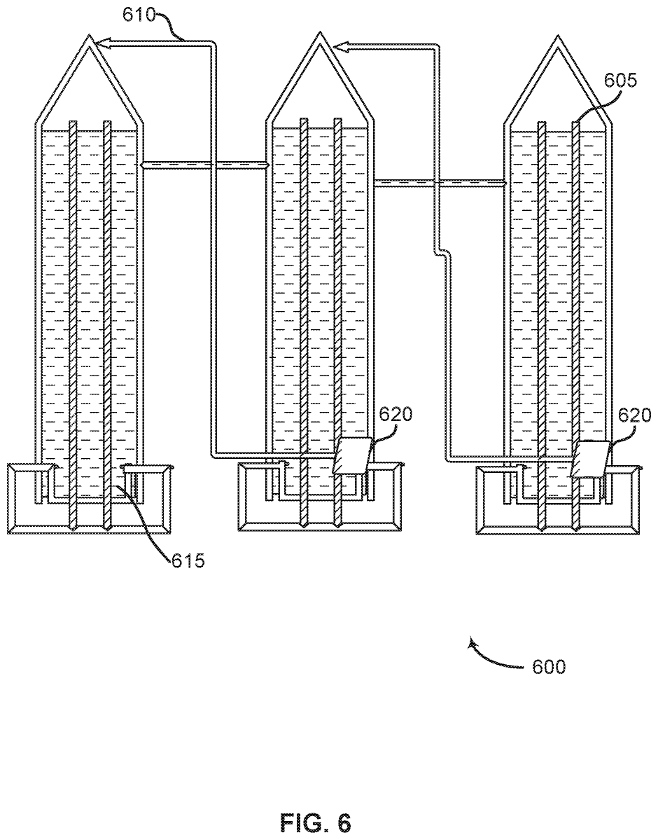

According to some aspects, lighting in FIG. 6 can be inside or outside of the transparent vessel to grow the organic precursor for bioplastic production and bacterial starch syrup along with other products that will be described in future patents.

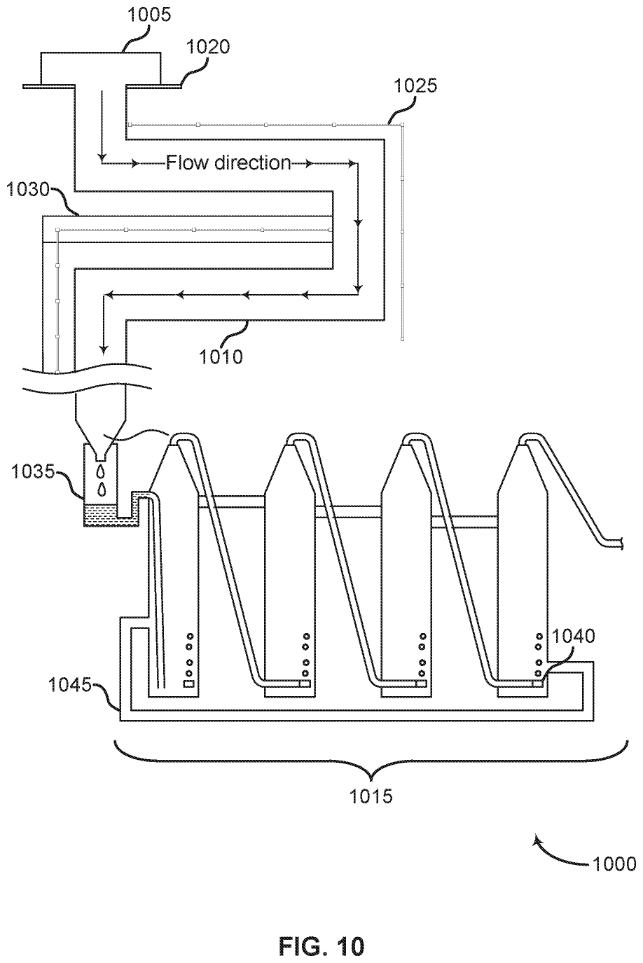

According to some aspects, lighting component 310 illuminates the algae with light. Lighting component 310 is an example of, or includes aspects of, the corresponding element described with reference to FIG. 10.

According to some aspects, aeration component 315 circulates liquid through the algae growth environment 305 and separates a gas oxygen and reuses carbon dioxide from air. According to some aspects, aeration component 315 is configured to separate a gas including oxygen from air and to diffuse the gas into a liquid circulating through the algae growth environment 305.

According to some aspects, separation component 320 uses the algae and ion exchange separation to condense or push the algae to the top of the tank, in a manner that does not harm the biomass from the algae growth environment 305. In some examples, separation component 320 uses anion exchange algae separation to separate the algae from a liquid.

According to some aspects, separation component 320 comprise an ion exchange component configured to separate the algae from a liquid by concentrating the algae at the top of the liquid.

One or more embodiments of the present disclosure include an anion exchange that facilitates a reaction. Ion exchange reaction is a reversible interexchange of charged particles (or ions). Ion exchange occurs when ions present in a medium are replaced with ions of a similar charge present in a matrix and the ions of the matrix are replaced by ions in the medium. In some cases, ion exchange sites are present throughout the matrix where functional groups of positively-charged ions (i.e., cations) or negatively-charged ions (i.e., anions) are fixed to the matrix network. The functional groups attract ions of an opposite charge to concentrate the algae in the water. Ion exchange systems use a bed of matrix including small and porous microbeads though a system, for example, a sheet-like mesh resin used for electrodialysis. The medium undergoes a chemical reaction to bind functional groups to the ion exchange sites located throughout the matrix. As a result, the medium is changed by the cross-linking matrix. The medium is restored for further use by a regeneration cycle after the medium is exhausted.

The matrix in the regenerant solution as the regeneration cycle is used to restore the matrix to full working condition. For example, regeneration cycle includes reversal of the reaction through the application of a concentrated regenerant solution such as a salt, an acid, or a caustic solution.

The ion exchange concentrates the algae at the top of the water. The ratio of the flow rate and the ion exchange material gives the ion exchange reaction time to concentrate the algae to the top of the tank.

According to some aspects, fermentation component 325 ferments the bacterial growth medium to produce an organic precursor compound including monomers or joined-together monomers. In some examples, fermentation component 325 ads a base to the bacterial growth medium to remove acid from the organic precursor compound.

According to some aspects, fermentation component 325 is configured to produce an organic precursor compound including monomers or joined-together monomers by adding heat and/or other chemical nutrients with the bacteria to the bacterial growth medium.

According to some aspects, extraction component 330 induces a chemical reaction to convert the organic precursor compound into a bioplastic material. In some examples, extraction component 330 performs a chemical lysing on the algae. In some examples, bacterial extraction component 330 performs a chemical lysing on bacteria in the bacterial growth medium. In some aspects, the bioplastic material includes polylactic acid (PLA), polyhydroxybutyalkanoates (PHA), polyhydroxybutyrates (PHB), or any combination thereof.

Embodiments of the present disclosure include an algae harvesting. The algae harvesting may include types of centrifugations, mechanical cell disruption and flocculation techniques. The aforementioned algae harvesting methods are not limited are examples of cell harvesting in water applications that are already described in other patents.

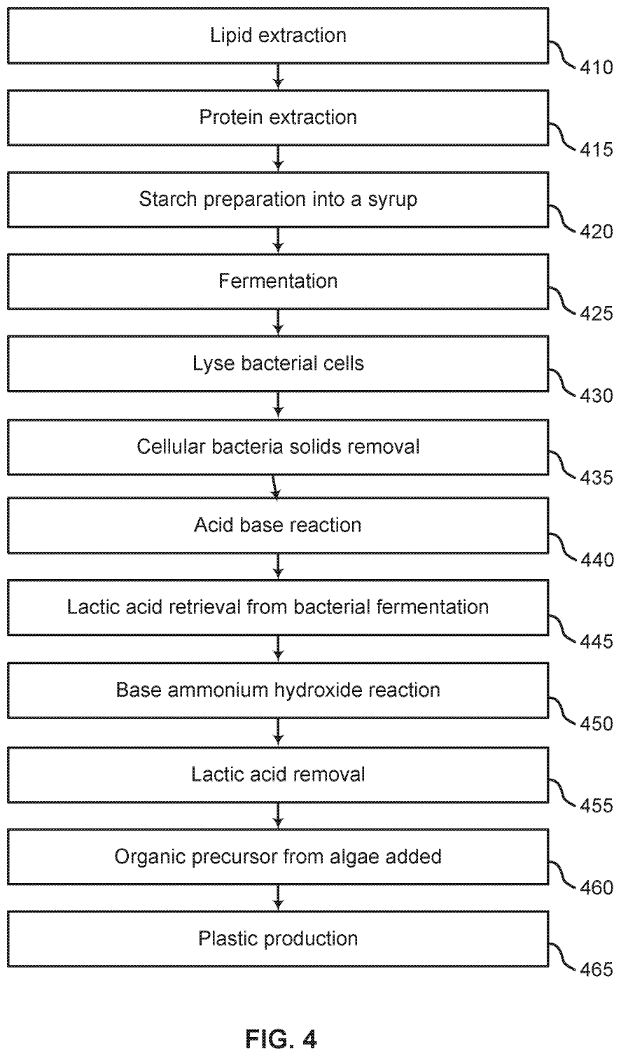

FIG. 4 shows an example of a method algal biomass pre-processing according to aspects of the present disclosure. In some examples, these operations are performed by a system including a processor executing a set of codes to control functional elements of an apparatus. Additionally or alternatively, certain processes are performed using special-purpose hardware. Generally, these operations are performed according to the methods and processes described in accordance with aspects of the present disclosure. In some cases, the operations described herein are composed of various substeps, or are performed in conjunction with other operations.

One or more embodiments of the present disclosure process feedstock into a biomass for bioplastic production. The processing of algal feedstock has many phases. The first phase is wet lipid extraction. The second phase is protein extraction by centrifugation. The third phase is sugar extraction, and it is the most important phase. The sugar extraction phase has the organic algae precursors present for the formation of biodegradable bioplastic present. Each phase can be put in any order to increase efficiency of production.

At operation 410, the system performs lipid extraction. An embodiment of the disclosure extract lipids from lysed algae cells using chemical or physical extraction techniques. Chemical extraction includes use of hexane to capture algae lipids. Alternatively, physical methods include pressurized processes using solvents to extract lipids from lysed or unlysed biomass.

At operation 415, the system performs protein extraction. The protein extraction may be performed by chemical and physical separation.

At operation 420, the system performs starch preparation into a syrup. One or more embodiments of the present disclosure include chemical catalytic decomposition of starch in a solution of water and salts. The enzymes and heat are used to facilitate the splitting of bonds in starch molecules to oligosaccharides and polysaccharides. The solution of water and salts may reach temperatures of 150-170° F. (i.e., approximately up to 72-75° C.) when heated. The boiling point of the solution is used at temperatures above 100° F. Additionally, the solution may be mixed continuously or intermittently.

In some cases, a hydrolysis reaction vessel may be configured to mix the solution by water bath and heat pumps or furnace as the mixture is heated. The time taken for enzymatic hydrolysis depends on the temperature of the solution and the concentration of the hydrolyzing agent. For example, a reaction may take place for about 12-24 hours or until the hydrolysis is completed. In the example of FIG. 4, steps 410, 415 and 420 can be processed in any order.

At operation 425, the system performs fermentation. An embodiment of the present disclosure includes a growth of bacteria and a fermentation method to test the type of bacterial growth for bioplastic production. Fermentation is a sterile process optimized according to the output or concentration of the products. Single or multiple strains of bacteria combined with the algae starch syrup is tested to optimize the production of lactic acid isomers, PHA, and PHB products. The parameters tested are temperature, sterility level, change of nutrient formulation with product output, and scalability. Bacterial fermentation to achieve appropriate product concentration lasts 12-24 hours.

At operation 430, the system performs lysing of bacterial cells. Lactic acid, PHA or PHB in bacteria is obtained by lysing or rupturing bacterial cell walls by heating the fermentation medium, mechanical disruption, homogenization, high frequency sound waves, extrusion, freeze-thaw cycles, and chemical lysis.

At operation 435, the system performs cellular bacteria solids removal. An embodiment of the present disclosure includes an apparatus for removal of cellular bacterial solids by clarifying the fermentation medium. Bacterial solids may be removed by gravity separation, centrifugation, and filtration of the fermentation medium.

At operation 440, the system performs a lactic acid extraction from the fermentation medium. One or more embodiments of the present disclosure include a chemical binding of lactic acid and cell lysis to release the lactic acid, PHA or PHB from the bacteria into the solution, then by adding a base to the fermentation medium. Alternatively, the amount of lactic acid is calculated for lactic acid removal from the fermentation medium using techniques such as color-changing chemical reactions, chromatography. For example, techniques for lactic acid determination include high-performance liquid chromatography (HPLC), nuclear magnetic resonance (NMR), infrared (IR), Fourier-transform infrared (FTIR), etc.

At operation 445, the system performs lactic acid retrieval from bacterial fermentation. An embodiment of the present disclosure uses Equation 1 a single replacement reaction for lactic acid removal. The single replacement reaction for lactic acid removal occurs when lactic acid and ammonium hydroxide are mixed at temperatures of 50-70° F. The quantity of ammonium hydroxide used to bind the lactic acid is a one-to-one ratio. For example, one lactic acid molecule uses one ammonium hydroxide molecule to produce one molecule of ammonium lactate.

1 NH₄OH+1 CH₃CH(OH)COOH=1 C₃H₉O₃N (1)

At operation 450, the system performs a base ammonium hydroxide reaction. One or more embodiments of the present disclosure include a calculation of the quantity of base to be added to the fermentation medium for lactic acid extraction and lysis of the bacterial cells. An assumed lactic acid concentration can substitute if the lactic acid concentration is not known. In some examples, the amount of lactic acid can be computed according to the equation: (Volume bacterial concentration) lactic acid %=X (amount of lactic acid). The amount of lactic acid in picograms can be converted to grams. The molar concentration of ammonium hydroxide added to the fermentation medium is equal to the molar concentration of lactic acid for a one-to-one reaction resulting in the formation of ammonium lactate. In an alternative embodiment, the system may also convert ammonium lactate to lactic acid. One or more embodiments of the present disclosure include splitting of bonds in ammonium ion using an acid for the formation of lactate.

At operation 455, the system performs lactic acid or lactate removal. Lactic acid is removed from the fermentation medium by adding an alcohol (e.g., isopropyl alcohol, ethanol, etc.). The alcohol separates the lactic acid from the water-based solution and the lactic acid settles on the bottom of the vessel.

At operation 460, the system performs algae precursor additions. The algae precursors are added to the lactic acid and then form a hard plastic polymer.

At operation 465, plastic production is performed.

FIG. 5 shows an example of a method 500 for an algae to bioplastic production method according to aspects of the present disclosure. In some examples, these operations are performed by a system including a processor executing a set of codes to control functional elements of an apparatus. Additionally or alternatively, certain processes are performed using special-purpose hardware. Generally, these operations are performed according to the methods and processes described in accordance with aspects of the present disclosure. In some cases, the operations described herein are composed of various substeps, or are performed in conjunction with other operations.

At operation 505, the system provides an algae growth environment configured to produce a bacterial growth medium by cultivating algae and algae plastic organic precursors. In some cases, the operations of this step refer to, or may be performed by, an algae growth environment as described with reference to FIGS. 3 and 8. In some cases, the system may perform PHAs and PHB collection. It is known in the prior art, and those skilled in the art, that existing collection methods can be used.

At operation 510, the system provides a fermentation component configured to produce an organic precursor compound including monomers or joined-together monomers by adding heat and bacteria to the bacterial growth medium. In some cases, the operations of this step refer to, or may be performed by, a fermentation component as described with reference to FIG. 3.

At operation 515, the system provides a plastic formulation and composition component configured to convert the organic precursor compound into a bioplastic material. In some cases, the operations of this step refer to, or may be performed by, a chemical conversion component. At operation 515, the algae precursors may be added to the lactic acid. The bioplastic formulation is lactic acid and algae precursors.

### Photobioreactor (PBR)

One or more embodiments of the present disclosure include an open tubular photobioreactor system. The system provides high efficiency of light energy utilization, low chances of contamination, and low moisture evaporation. Additionally, the system includes gas exchange, mixing of water and low impact by external environmental elements. The light utilization is high due to a light emitting diode (LED) system placed outside the tubular reactor. Additionally, controlled and constant environmental conditions are maintained in an indoor multi-story facility to ensure the environmental impact is minimum. The contamination is controlled using water processing and cultivation methods. The photobioreactor operates in an indoor temperature-controlled warehouse style building. Direct sun light is prevented.

One or more embodiments of the present disclosure include a method of operating an open photobioreactor for culturing photosynthetic microorganisms, wherein the photobioreactor comprises a culture solution. Additionally, the wall of the photobioreactor suitable for containing the culture medium comprises a waterproof and ridged material. In some cases, the waterproof and ridged material is a thin or a thick polymer-based material for culturing photosynthetic microorganisms, wherein the photobioreactor is suitable for containing a culture solution. The food grade materials suitable for use in the photobioreactor material include a translucent or opaque structural rigidity.

One or more embodiments of the disclosure include cultivation in the vertical position with a flexible configuration of the photobioreactor design. The reactor set up can be placed in any geographical location. The photobioreactor system reduces the risk of contamination.

One or more embodiments of the present disclosure include an apparatus to produce biomass. Biomass may be produced from algae using photosynthesis by LED lights in the photobioreactor. The LED lights use specific wavelengths for cultivation and algae growth. A percentage of wavelengths of light in the visible spectrum is used to grow individual strains of algae.

In an embodiment, CO₂ gas mixture is bubbled through a culture medium by a tube or tube-like device that extends into the culture medium at the bottom of the photobioreactor. Bubble size affects CO₂ absorption, i.e., smaller bubble size increases CO2 absorbed in the water.

Non-toxic algae may be used as a feedstock. For example, green algae from the class Chlorophyta are used in a commercial setting and are not toxic species of algae. In some cases, algae that produce high concentrations of polysaccharides may be used. An embodiment of the present disclosure includes a tubular reactor that is attached to a support structure connected to the floor and the tubes. In some cases, the tubes are attached to a metal support system to keep the tubes in place. The design of the algae photobioreactor includes a clear food grade tube, caps, CO₂ enriched air and water lines, LED lighting and a sampling port.

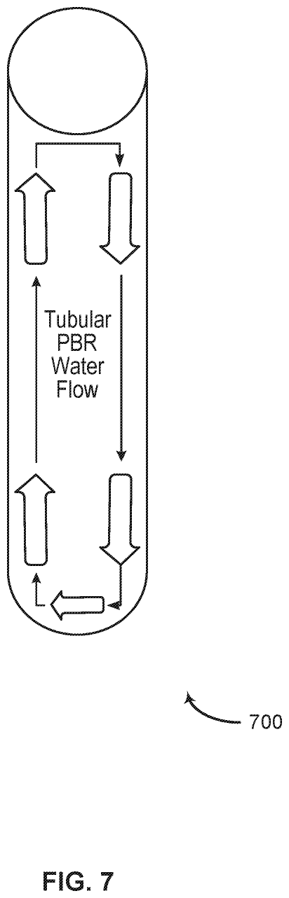

FIG. 6 shows an example of a configuration of tubes 600. Tubes 600 is an example of, or includes aspects of, the corresponding element described with reference to FIGS. 7, 8, 9 and 10. In one aspect, tubes 600 includes lighting 605, gas recycling tubes 610, and algae growth environment 615. Lighting 605 may be an incandescent lamp, compact fluorescent, halogen lamps, metal halide, light emitting diode (LED). fluorescent tube. neon lamps, high intensity discharge (HID), or the like. The gas recycling tubes 610 move gases that form in the algae growth environment 615, which enters into the gas intake 620. In some cases, additional lighting may be arranged on the outside of the tubes 600 in a vertical orientation, mounted a separate lighting structure.

The algae growth environment 615 is configured to produce a bacterial growth medium and organic algae plastic precursors by cultivating algae. Algae growth environment 615 is an example of, or includes aspects of, the corresponding element described with reference to FIG. 3.

FIG. 7 shows an example of water flow diagram according to aspects of the present disclosure. The lighting components, with reference to FIG. 6, are present outside the tube. The gas-CO₂ mixture flows down and releases at the bottom and returns to the top of the tube using the space. Tube 900 is an example of, or includes aspects of, the corresponding element described with reference to FIGS. 6, 9, and 10.

FIG. 8 shows an example of algae condensing diagram according to aspects of the present disclosure. The example shown includes first tank 800, filter 805, and second tank 80. According to an embodiment of the disclosure, algae may be passed from the first tank 800 through the filter 805 to a second tank 810.

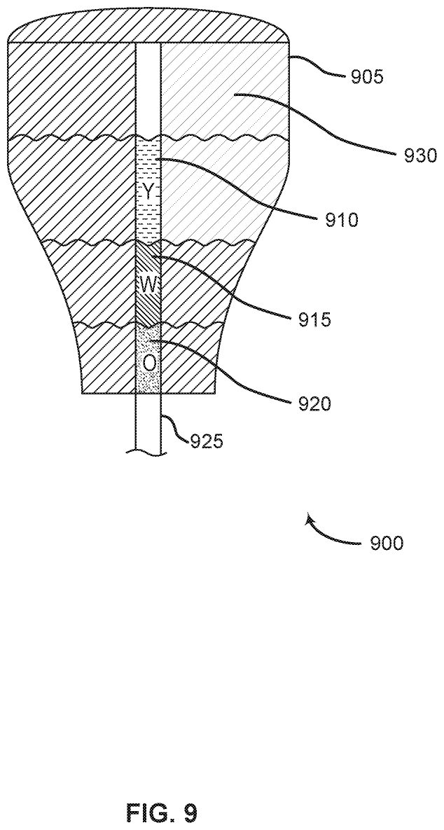

FIG. 9 shows an example of liquid/liquid separation tank 900 according to aspects of the present disclosure. In one aspect, liquid/liquid separation tank 900 includes housing 905, broth 910, alcohol 915, lactic acid 920, tube 925, and water 930.

One or more embodiments of the present disclosure include a PBR system for data processing that is monitored in real time by sensors, automated robots and collaborative robots that are intended for direct human interaction (i.e., cobots). The process controller maintains optimal growth parameters in the photobioreactor system using data points received by the sensors.

FIG. 10 shows an example of an algae cultivation unit 1000 according to aspects of the present disclosure. The algae cultivation unit 1000 includes one or more algae tanks 1005, tubes 1010, and photobioreactors 1015.

Algae tank 1005 may be placed on an oscillating platform 1020 to agitate or mix the algae and water. The algae tank 1005 may be open on the top and may be made of any suitably clear material. In one aspect, one or more algae tank 1005 may be used. In some cases, the algae tank 1005 is set up in an array formation and each algae tank feeds a different tube 1010.

Tubes 1010 may be arranged in one or more folds, with a light blocking barrier 1030 between each fold. The tubes may be gravity fed and filled with algae and water from the algae tank 1005. In one aspect, tubes 1010 may be made of metal or any suitable material. The algae mixture provided through the tubes 1010 through the series of folds or bends. A lighting component 1025 is located between each horizontal part of the tube. In some cases, a sample port with be present in the tubes 1010 to extract a sample of the materials.

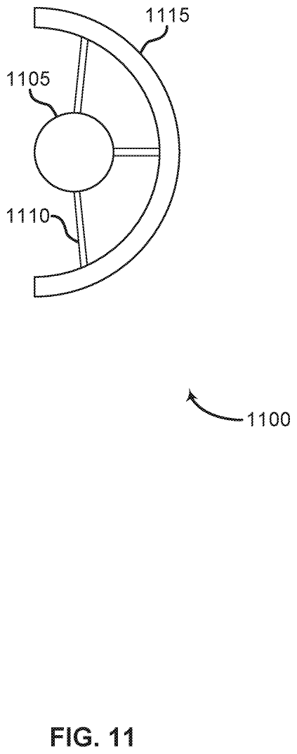

The lighting component 1025 may be surrounded by a light blocking barrier 1030 which may be used to prevent light from shining on multiple tubes. In some cases, the light component 1025 and light blocking member 1030 may be included in a light module, with reference to FIG. 11. The lightning component 1025 may include different lights with different wave lengths or different intensities. For example, the lighting component 1025 may be of a higher intensity at the top of the photobioreactor and a lower intensity at the bottom of the photobioreactor. In various examples, the intensities may vary. Light blocking barrier 1030. is an example of, or includes aspects of, the corresponding element described with reference to FIG. 11. The lighting component may be an incandescent lamp, compact fluorescent, halogen lamps, metal halide, light emitting diode (LED). fluorescent tube. neon lamps, high intensity discharge (HID), or the like.

Photobioreactors 1015 may be set up in an array formation. For example, the array may be 11 photobioreactors long and 15 photobioreactors wide. Algae and water from the tubes 1010 are provided to a reservoir 1035, which then provides the algae and water to the photobioreactors 1015. In one aspect, photobioreactors 1015 include a gas collection chamber 1040 and an electrical shock system 1045. The gas collection chamber 1040 may provide gases such as air, CO2, and others or the like to the bottom of the photobioreactors 1015. The electrical shock system 1040 may move water with the use of indirect pressure from the water gas collection chamber 1040 and pressurized air.

Gasses may be injected into the algae and water at the bottom of the photobioreactor at the gas collection chamber 1040. The air is captured on the top of the cone of the photo-bioreactor and pressure moves the air from the top of the cone to the bottom of the next photobioreactor. The concentration of CO2 is reduced each time the CO2 moves from tank to tank until the CO2 is absorbed by the algae.

FIG. 11 shows an example of a light blocking barrier 1100 according to aspects of the present disclosure. The light blocking barrier 1100 may be used to prevent light from shining on multiple tubes. In some cases, the light blocking barrier 1100 is comprised of metal, plastic, or any suitable material to prevent light from passing through.

The light blocking barrier 1100 includes light module 1105, which is surrounded by light blocking member 1115. The light module 1105 and light blocking member 1115 are connected to each other using supports 1110. Light blocking barrier 1100. is an example of, or includes aspects of, the corresponding element described with reference to FIG. 10.

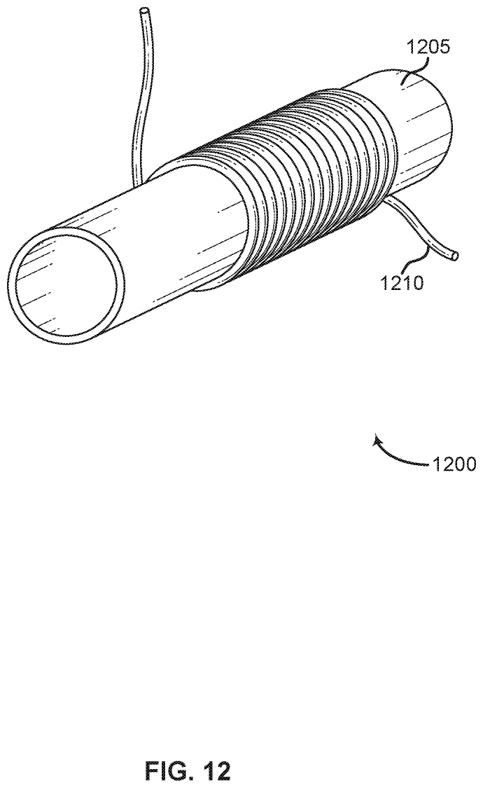

FIG. 12 shows an example of a deionization apparatus 1200 according to aspects of the present disclosure. In some embodiments, Algae is passed through a redox reaction that separates the water from the algae. A redox reaction occurs when the algae interact with the anion exchange in the deionization apparatus 1200 causing a chain reaction that moves the algae away to the top of the water. The ionized algae can be harvested or deionized to continue growth.

In one embodiment, the deionization apparatus 1200 includes a conductive coil 1210 (e.g., a copper coil) wrapped around a pipe 1205 (e.g., a lead pipe). In some cases, the conductive coil 1210 is attached to an AC power source, creating a magnetic field that pulls off ions from the algae.

The description and drawings described herein represent example configurations and do not represent all the implementations within the scope of the claims. For example, the operations and steps may be rearranged, combined or otherwise modified. Also, structures and devices may be represented in the form of block diagrams to represent the relationship between components and avoid obscuring the described concepts. Similar components or features may have the same name but may have different reference numbers corresponding to different figures.

Some modifications to the disclosure may be readily apparent to those skilled in the art, and the principles defined herein may be applied to other variations without departing from the scope of the disclosure. Thus, the disclosure is not limited to the examples and designs described herein, but is to be accorded the broadest scope consistent with the principles and novel features disclosed herein.

In this disclosure and the following claims, the word “or” indicates an inclusive list such that, for example, the list of X, Y, or Z means X or Y or Z or XY or XZ or YZ or XYZ. Also the phrase “based on” is not used to represent a closed set of conditions. For example, a step that is described as “based on condition A” may be based on both condition A and condition B. In other words, the phrase “based on” shall be construed to mean “based at least in part on.” Also, the words “a” or “an” indicate “at least one.”

1. A method comprising:
cultivating or harvesting algae to produce starch for a bacterial growth medium and biphytoplankton extract, wherein the bacterial growth medium includes glucose or fructose;
fermenting the bacterial growth medium to produce an organic precursor compound including monomers or joined-together monomers; and
adding the biphytoplankton extract to the organic precursor compound to induce a chemical reaction to convert the organic precursor compound into a bioplastic material.

2. The method of claim 1, further comprising:
removing toxic algae from a water source to obtain the algae.

3. The method of claim 1, further comprising:
producing the algae in an algae growth environment;
illuminating the algae with light; and
circulating liquid through the algae growth environment.

4. The method of claim 3, further comprising:
separating a gas including carbon dioxide from air;
diffusing the gas into the liquid prior to circulating the liquid through the algae growth environment.

5. The method of claim 1, further comprising:
using anion exchange algae separation to separate the algae from a liquid.

6. The method of claim 1, further comprising:
hydrolyzing the algae to produce the bacterial growth medium and to produce algae precursors including the starch.

7. The method of claim 1, further comprising:
performing a chemical lysing on the algae.

8. The method of claim 1, further comprising:
extracting lipids using a pressure chemical extraction process.

9. The method of claim 1, further comprising:
separating protein from the algae using chemical and physical separation.

10. The method of claim 1, further comprising:
performing a chemical lysing on bacteria from in the bacterial growth medium.

11. The method of claim 1, further comprising:
removing solids from the bacterial growth medium using a mechanical separation.

12. The method of claim 1, further comprising:
adding a base to the bacterial growth medium to make ammonium lactate.

13. The method of claim 1, further comprising:
separating the organic precursor compound from the fermented bacterial growth medium using an alcohol.

14. The method of claim 1, further comprising:
the bioplastic material comprises polylactic acid (PLA) with algae precursors, polyhydroxybutyalkanoates (PHA), polyhydroxybutyrates (PHB), or any combination thereof.

15. The method of claim 1, further comprising:
extracting an algae precursor to form a bioplastic material having a molecular shape of a helix.

16. An apparatus comprising:
an algae growth environment configured to produce a bacterial growth medium and algae precursors by cultivating algae;
a fermentation component configured to produce an organic precursor compound including monomers or joined-together monomers by adding heat and bacteria to the bacterial growth medium; and
a chemical conversion component configured to convert the organic precursor(s) compound into a bioplastic material.

17. The apparatus of claim 16, wherein:
the algae growth environment comprises a plurality of transparent tubes. The plurality of tubes is used in many different configurations.

18. The apparatus of claim 16, further comprising:
a light-emitting diode (LED) disposed on the algae growth environment, and configured to provide light to the algae.

19. The apparatus of claim 16, further comprising:
one or more check valves orientations disposed on the algae growth environment and configured to release air gas from the algae growth environment.

20. The apparatus of claim 16, further comprising:
an aeration component configured to separate a gas including carbon dioxide from air and to diffuse the gas into a liquid circulating through the algae growth environment.

21. The apparatus of claim 16, further comprising:
an ion separation component configured to separate the algae from a liquid by concentrating the algae at the top of the liquid.

22. The apparatus of claim 16, further comprising:
a separation component configured to extract lipids or proteins from the algae.
